(19) United States
(12) Patent Application Publication
(10) Pub. No.: US 2014/0051970 A1
(43) Pub. Date: Feb. 20, 2014

(54) OBJECT INFORMATION ACQUIRING APPARATUS

(71) Applicants: Hisafumi Ebisawa, Tokyo (JP); Haruo Yoda, Nishitama-gun (JP); Kenichi Nagae, Yokohama-shi (JP)

(72) Inventors: Hisafumi Ebisawa, Tokyo (JP); Haruo Yoda, Nishitama-gun (JP); Kenichi Nagae, Yokohama-shi (JP)

(73) Assignee: CANON KABUSHIKI KAISHA, Tokyo (JP)

(21) Appl. No.: 14/113,614

(22) PCT Filed: Apr. 26, 2012
(86) PCT No.: PCT/JP2012/002874

§ 371 Date: Oct. 24, 2013

(30) Foreign Application Priority Data
May 10, 2011 (JP) 2011-105317

(51) Int. Cl. A61B 5/00

(52) U.S. Cl. CPC A61B 5/0095
USPC 600/407

(57) ABSTRACT

The present invention employs an object information acquiring apparatus comprising a probe including a plurality of conversion elements which receive acoustic waves emitted from an object and convert the acoustic waves into received signals, a delay unit which matches phases of the plurality of received signals output from the plurality of conversion elements, a signal adder which adds the plurality of received signals output from the delay unit, for each group to obtain latter input signals, and an adaptive signal processor which generates internal image data of the object by performing adaptive signal processing on the plurality of latter input signals output from a plurality of the signal adders.

### TECHNICAL FIELD

The present invention relates to an object information acquiring apparatus which receives acoustic waves emitted from an object and performs adaptive signal processing on the received signals.

### BACKGROUND ART

As technology for acquiring biological information in an object, there is a method of transmitting and receiving ultrasound waves to and from an object and thereby generating biological image information (for instance, a cross-sectional image or a three-dimensional image) based on the signals obtained from the received ultrasound echoes, and this method is being used in many practical applications. Note that the ultrasound echoes are the ultrasound waves reflected from the object.

The processing system of an ultrasound imaging apparatus for performing linear scanning is now explained with reference to FIG. 2 and FIG. 3. FIG. 2 is a schematic diagram of the system of a general ultrasound device. The ultrasound device comprises a transmitting circuit processing system 001, a receiving circuit processing system 002, a system controller 003, an ultrasound probe 004, a received signal processor 010, an image processor 007, and an image display unit 008. The ultrasound probe 004 comprises a plurality of conversion elements (transducers) 005. FIG. 3 shows the details of the receiving circuit processing system 002 and the received signal processor 010, and the receiving circuit processing system. 002 is configured from a delay unit 009 (delay unit), and the received signal processor 010 is configured from an adder 018, a Hilbert transformer 012, and a quadrature detector 020.

The linear scanning operation of the ultrasound imaging apparatus is now explained. When creating image data on an image scanning line 019 at an arbitrary position on a plurality of conversion elements 005 aligned linearly, transmitting apertures are formed around the arbitrary position by using the plurality of conversion elements 005 positioned symmetrically. Ultrasound waves are transmitted from the conversion elements 005 configuring the transmitting apertures, and a transmitting beam is formed on the image scanning line 019 at the arbitrary position. Normally, the direction and position of the transmitting beam are set so as to correspond to the position of the intended image scanning line 019 within the imaging target. The transmitting beam is formed by performing delay processing on the transmission timing from the respective conversion elements 005 configuring the transmitting apertures, and causing them to focus at the target depth. Conditions are set in the system controller 003 for the foregoing series of operations, and these operations are executed by the transmitting circuit processing system 001.

The transmitting beam formed as described above is reflected and scattered in the object.

The ultrasound echoes from within the object are received by the receiving apertures formed from a plurality of conversion elements 005 positioned symmetrically around the arbitrary position, and converted into electrical signals in the respective conversion elements 005. Since the ultrasound echoes from within the object also reach the conversion elements as noise from positions other than the intended position, synthesis processing of extracting signals only from the intended direction and position is executed in the receiving circuit processing system 002 and the received signal processor 010.

Delay-and-sum processing is adopted as the general synthesis processing in the ultrasound imaging apparatus. The delay-and-sum processing is now explained with reference to FIG. 3. The delay-and-sum processing is processing of the delay unit 009 performing delay processing on the ultrasound echoes from the intended object that are received from the respective conversion element 005 in asynchronous timing. As a result of matching the phases of the respective received signals based on the foregoing processing and further adding these with the adder 018, noise components other than the intended signals are negated and suppressed.

The signals that were subject to synthesis processing are converted into complex signals by the Hilbert transformer 012, and subsequently subject to envelope detection processing by the quadrature detector 020 and then output.

The data group in which this output value is calculated for each time series is the image scanning line data (depth direction) at the arbitrary position. As a result of repeating the foregoing series of transmission and reception processing by moving the transmitting apertures and the receiving apertures in the linear direction, two-dimensional image information is output. A B-mode image of the object is created by subjecting the image information to LOG compression or the like in the image processor 007, and this is output to the image display unit 008 (FIG. 2).

The processing system of an ultrasound imaging apparatus using general delay-and-sum processing is as described above.

Meanwhile, as technology that was developed in the field of wireless radar, there is adaptive signal processing which uses a plurality of array antennas. The adaptive signal processing is a method of efficiently calculating, as a power value, the signals from the intended direction among the signals that are received from the respective antennas. As described above, the ultrasound imaging apparatus performs synthesis processing of extracting signals only from the intended direction and position in the receiving circuit processing system 002 and the received signal processor 010 of FIG. 2. Meanwhile, since the adaptive signal processing is also characterized in that it is performed for efficiently calculating the intended signals, a system of causing the receiving circuit processing system 002 and the received signal processor 010 to be compatible with adaptive signal processing can be considered.

The processing of Directionally Constrained Minimum Power, which is a type of adaptive signal processing, is now explained.

Considered is a linear array antenna configured from M number of conversion elements. Among the M number of array antenna elements, set the signal received by the k-th antenna element at time t as xk(t), and set the group of the M number of signals as X (t). The representation of this as a matrix is shown in Formula (1).

### [Math. 1]

X(t)=[x₁(t),x₂(t), . . . ,xₘ(t)]^{T} (1)

Note that T represents a transposed matrix.

In order to calculate the output from the respective signals of this array signal group, the complex weight vector W needs to be calculated. The weight vector W and the output y (t), and the rule for obtaining the output power Pout are shown in Formulas (2) to (4) below.

### [Math. 2]

W=[w₁,w₂, . . . ,w_{M}]^{T} (2)

y(t)=W^{H}X₍ₜ₎=X₍ₜ₎^{T}W* (3)

Pout=1/2E[|y(t)|²]=½W^{H}RxxW (4)

Note that H represents the complex conjugate transpose, and the superscript * represents the complex conjugate.

Rxx used in the foregoing rule is the covariance matrix of the received signal X(t), and is as shown in Formula (5).

### [Math. 3]

Rxx=E[X₍ₜ₎X₍ₜ₎^{H}] (5)

Note that E represents the time average.

In this adaptive signal processing, the weight vector W is adaptively changed and processed so as to optimize the output signal.

The expression “to optimize” means to minimize the output value in a state where the sensitivity of the direction of the intended signal is constrained to 1, and the problem is formulated as shown in Formula (6) and Formula (7) below.

$$\begin{matrix}[ Math . 4 ] & \\ {min}_{W} ( P out = \frac{1}{2} {W}^{H} RxxW ) & ( 6 ) \\ subject to {W}^{H} a ( θ ) = 1 & ( 7 )\end{matrix}$$

As a result of solving this formulated problem, the optimal weight vector Wcp can be calculated as shown in Formula (8) below. Based on this weight vector Wcp, noise signals from a direction other than the direction of the intended signals can be suppressed to the maximum extent.

$$\begin{matrix}[ Math . 5 ] & \\ Wcp = \frac{{Rxx}^{- 1} a ( θ )}{{a}^{H} {Rxx}^{- 1} a ( θ )} & ( 8 )\end{matrix}$$

As a result of using this optimal weight vector Wcp, the optimal output power of the array is transformed to Formula (9) shown below.

$$\begin{matrix}[ Math . 6 ] & \\ P out = \frac{1}{2 {a}^{H} ( θ ) {Rxx}^{- 1} a ( θ )} & ( 9 )\end{matrix}$$

The basic power conversion method of the Directionally Constrained Minimum Power is as described above.

### CITATION LIST

### Non Patent Literature

[NPL 1]
IEEE Trans Ultrason Ferroelectr Freq Control. 2007 August; 54(8): 1606-13
[NPL 2]
IEEE Trans Ultrason Ferroelectr Freq Control. 2009 October; 56(10): 2187-97

### SUMMARY OF INVENTION

### Technical Problem

As explained above, as a result of applying the adaptive signal processing to the received signals, an image having a superior azimuth resolution can be reconstructed in comparison to the conventional image reconstruction based on the delay-and-sum processing (Non Patent Literature 1). Nevertheless, with the adaptive signal processing, it is known that the processing volume in the course of calculating the inverse matrix Rxx⁻¹ upon power conversion becomes enormous. Thus, if the adaptive signal processing is simply diverted to an ultrasound imaging apparatus, the processing volume of the apparatus becomes an unrealistic level as medical equipment. In order to resolve the foregoing problem, proposed is a system of combining the input signals to the adaptive signal processing in advance so as to reduce the number of signals (Non Patent Literature 2).

In the foregoing proposal, the received signals are acquired collectively, the signals are classified by spatial frequency by the discrete Fourier transform (DFT) processing, and, after integrating the plurality of signals, adaptive signal processing is performed thereto. Nevertheless, relatively complex processing steps such as DFT processing and grouping for each spatial frequency need to be provided in the former processing. Moreover, if the number of received signals is increased in order to achieve a higher resolution in the future, the foregoing former processing steps will also increase. Thus, in light of the overall system of the ultrasound imaging apparatus, it cannot be said that the foregoing problem has been completely resolved.

Generally speaking, what are important in an ultrasound imaging apparatus is real-time processing and shortening of the measurement time. Accordingly, it is important to suppress the volume of adaptive signal processing by using a simple system with a small processing volume, and provide a high definition ultrasound imaging apparatus of a realistic size as the overall system.

The processing flow and volume of the adaptive signal processing are now explained in detail.

The processing system of an ultrasound imaging apparatus for performing linear scanning using the adaptive signal processing is now explained with reference to FIG. 1 and FIG. 4. FIG. 1 shows an example where the received signal processor 010 is replaced by the adaptive signal processor 006 in the system of the general ultrasound device shown in FIG. 2. FIG. 4 shows the details of the receiving circuit processing system 002 and the adaptive signal processor 006. The receiving circuit processing system 002 is configured from the delay unit 009, and the adaptive signal processor 006 is configured from the Hilbert transformer 012, the covariance matrix calculator 013, the spatial smoothing calculator 014, and the electric power calculator 015.

Here, an example of the CAPON method as one type of Directionally Constrained Minimum Power of the adaptive signal processing is explained.

Let it be assumed that the receiving apertures are formed using M number of conversion elements 005. The Hilbert transformer 012 performs Hilbert transformation to M channel signals in which their phases are matched by the delay unit 009. An M by M covariance matrix is created by the covariance matrix calculator 013. Subsequently, the foregoing covariance matrix is transformed into an N by N submatrix by the spatial smoothing calculator 014. The number of rows (N) after the spatial averaging is preferably around half of the number of input channels (M) (N approximately equals to M/2). Nevertheless, the CAPON method will function reasonably so as long as the relation of M>N>=2 is achieved. The electric power calculator 015 adaptively calculates the optimal power based on the inverse matrix of the N by N submatrix and the constrained vector related to the signal arrival direction. Here, as a result of setting the signal arrival direction to the transmitting beam direction, performed is processing of preferentially selecting the signals of the ultrasound echoes from the transmitting beam direction in comparison to the signals of the ultrasound echoes from other directions. It is thereby possible to enable synthesis processing that can achieve a superior orientation direction resolution.

The output calculated by the CAPON method is subject to LOG compression or the like in the image processor 007, a B-mode image of the object is created as with the general delay-and-sum processing, and this is output to the image display unit 008 (FIG. 1).

Nevertheless, with the adaptive signal processing including the CAPON method, the processing volume tends to increase relative to the number of inputs. Among the above, during the process of calculating the inverse matrix Rxx⁻¹ of the power converter, the processing volume thereof corresponds to a cube of the number of rows. For example, assuming that the processing volume for calculating the power from a 16 by 16 sized matrix as with the foregoing simulation is 4096 L, the processing volumes in cases where the matrix size is 8 by 8 and 4 by 4 are respectively 512 L and 64 L. In the case of ultrasound image reconstruction which requires numerous input signals, the processing volume will become enormous if the adaptive signal processing is simply applied. Real time display is difficult with the foregoing processing volume and, since the scan time will also increase upon acquiring the volume data, the subject's physical and psychological burden cannot be ignored. This factor is a major challenge for realizing practical application.

The present invention was devised in view of the foregoing problems. Thus, an object of this invention is to provide an object information acquiring apparatus capable of configuring an ultrasound image having a high azimuth resolution while suppressing the processing volume.

### Solution to Problem

The present invention provides an object information acquiring apparatus, comprising:

a probe including a plurality of conversion elements which receive acoustic waves emitted from an object and convert the acoustic waves into received signals;

a delay unit which matches phases of the plurality of received signals output from the plurality of conversion elements;

a signal adder which groups the plurality of received signals output from the delay unit, and adds the received signals for each of the groups to obtain latter input signals; and

an adaptive signal processor which generates internal image data of the object by performing adaptive signal processing on the plurality of latter input signals output from the signal adder.

### Advantageous Effects of Invention

According to the present invention, it is possible to provide an object information acquiring apparatus capable of configuring an ultrasound image having a high azimuth resolution while suppressing the processing volume.

Further features of the present invention will become apparent from the following description of exemplary embodiments with reference to the attached drawings.

### BRIEF DESCRIPTION OF DRAWING

FIG. 1 is a block diagram of the ultrasound imaging apparatus of the present invention.

FIG. 2 is a block diagram of a general ultrasound imaging apparatus.

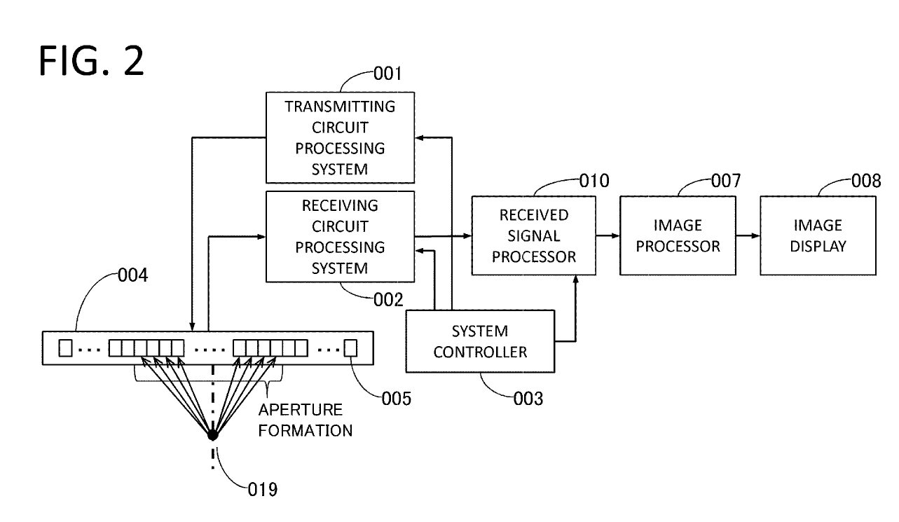

FIG. 3 is a block diagram of an image configuration using the delay-and-sum processing.

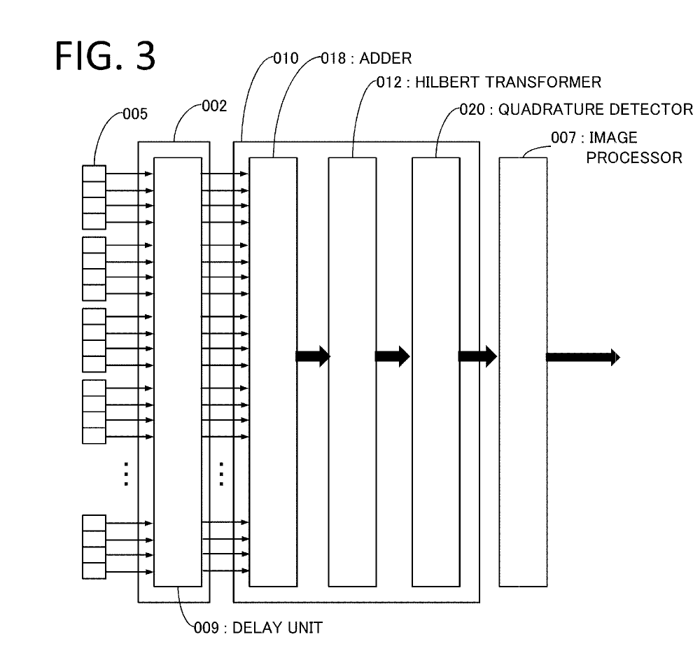

FIG. 4 is a block diagram of an image configuration using the adaptive signal processing.

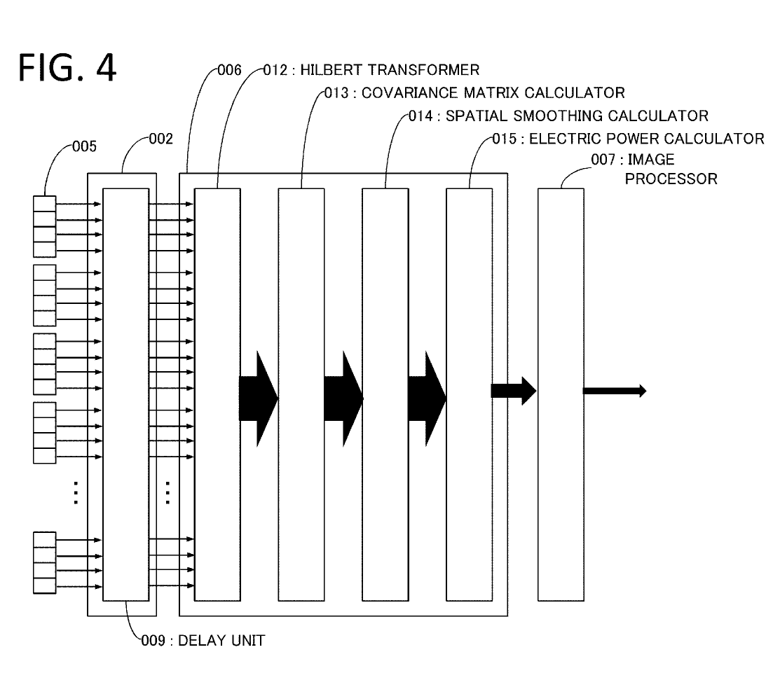

FIG. 5 is a block diagram of the configuration of Embodiment 1.

FIG. 6 is a block diagram of the configuration of Embodiment 2.

FIG. 7 is a block diagram of the configuration of Embodiment 3.

FIG. 8 is a block diagram of the configuration of Embodiment 4.

FIG. 9 is a diagram showing a simulation image for explaining the effect of the embodiments.

FIG. 10 is a diagram showing a simulation cross-sectional image for explaining the effect of the embodiments.

FIG. 11 are diagrams showing simulation image for explaining the effect of Embodiment 1.

FIG. 12 is a diagram explaining the effect of Embodiment 2.

FIG. 13 are diagrams explaining the effect of Embodiment 3.

### DESCRIPTION OF EMBODIMENTS

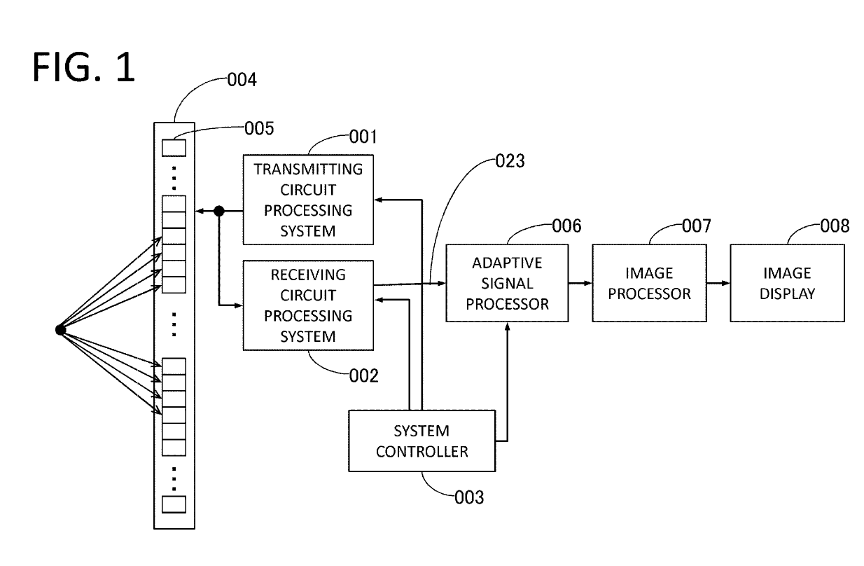

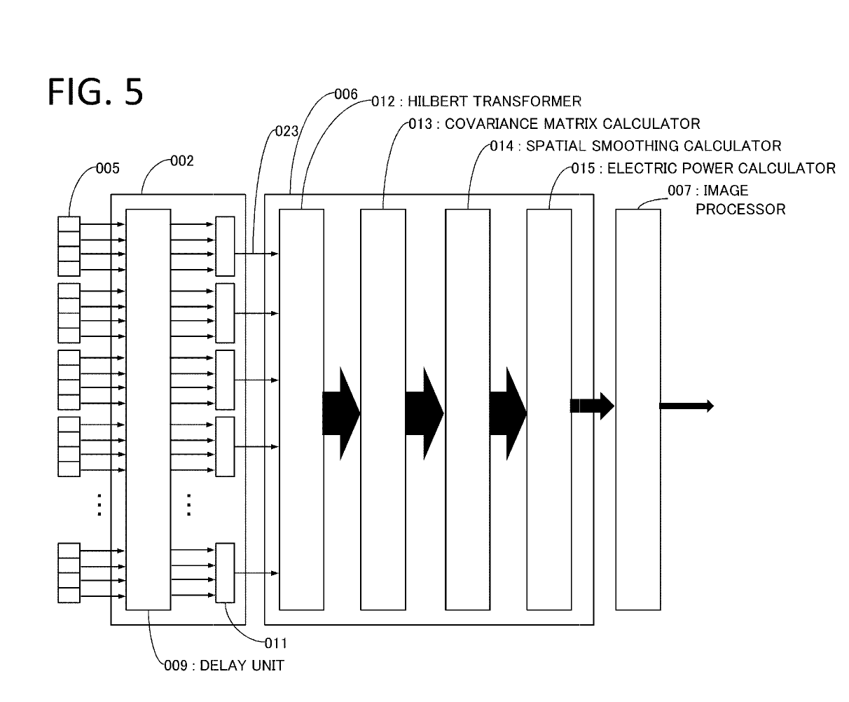

The preferred embodiments of the present invention are now explained with reference to FIG. 1 and FIG. 5.

The ultrasound imaging apparatus of the present invention is an apparatus which receives and processes ultrasound echoes from an object, and thereby acquires biological information as image information (image data). The biological image information can be presented not only as a tomographic image, but also as a three-dimensional image. The ultrasound imaging apparatus is mainly configured from a transmitting circuit processing system which irradiates the biological object with ultrasound waves, and a received signal processor which receives the reflected waves of the transmitted signals and configures images. Note that the receiving processor is configured from the receiving circuit processing system 002 and the adaptive signal processor 006 of FIG. 1.

The ultrasound imaging apparatus of the present invention also includes an apparatus which transmits ultrasound waves to the object and uses the foregoing ultrasound echo technology. In addition to the above, the ultrasound imaging apparatus of the present invention includes an apparatus which receives acoustic waves generated in the object by irradiating the object with light (electromagnetic waves), and uses the photoacoustic effect of acquiring object information as image data. Accordingly, the ultrasound imaging apparatus of the present invention can also be referred to as an object information acquiring apparatus. When the object is a biological object, the object information acquiring apparatus can also be referred to as a biological information acquiring apparatus. Here, acoustic waves are typically ultrasound waves, and include elastic waves referred to as sound waves, ultrasound waves, photoacoustic waves, and optical ultrasound waves.

With the former object information acquiring apparatus that uses the ultrasound echo technology, object information is information which reflects the differences in the acoustic impedance of the tissues inside the object. With the latter object information acquiring apparatus that uses the photoacoustic effect, object information shows the generation source distribution of the acoustic waves generated by optical irradiation, the initial sound pressure distribution in the object, the light energy absorption coefficient density distribution that is derived from the initial sound pressure distribution, the absorption coefficient distribution, or the concentration distribution of the substance configuring the tissues. The substance concentration distribution is, for example, oxygen saturation distribution or oxidized/reduced hemoglobin concentration distribution. The foregoing object information is also image data for generating internal images of the object through reconstruction based on the foregoing information.

Since the feature of the present invention is related to a received signal processor and the transmission process of the ultrasound waves is the same as the transmission processing of general ultrasound devices described above, the detailed explanation concerning the transmission of signals is omitted. As the characteristic configuration of the present invention, foremost, several of the received signals are respectively subject to delay-and-sum processing and combined in advance in the delay unit 009 and the sum processor 011 of the received signal processing system 002. Subsequently, the adaptive signal processor 006 performs adaptive signal processing by using the latter input signal 023 that was integrated by the sum processor, and thereby reconstructs the image of biological information. The received signals are signals in which the ultrasound echoes from the object based on the transmitting beam are received by the respective conversion elements 005 configuring the receiving apertures.

As a result of combining several signals in advance as described above, improvement of the azimuth resolution can be realized in adaptive signal processing of a realistic volume. Moreover, since this former system is basically a result of simply adding a plurality of sum processors 011, simple and small-volume former processing can be realized. Based on the above, it is possible to realize a high definition ultrasound imaging apparatus with a realistic processing volume throughout the entire system.

The adaptive signal processing is a method that is mainly used in the field of radars for adaptively changing, according to the signals, the weight coefficient (weight vector) upon synthesizing the received signals obtained with a plurality of conversion elements in order to improve the sensitivity of the intended observation direction. There are several processing methods for the foregoing adaptive signal processing depending on the technique thereof, but in this embodiment the CAPON method as one type of Directionally Constrained Minimum Power (DCMP) is adopted. Note that other types of adaptive signal processing such as the Multiple Signal Classification or Estimation of Signal Parameters via Rotational Invariance Techniques (MUSIC method, ESPRIT method) may also be used.

In the adaptive signal processing, what is important is the suppression of covariance of the intended wave and noise. However, signals of ultrasound waves tend to have higher covariance of the intended wave and noise in comparison to signals of radars. Thus, it is necessary to additionally combine a method referred to as the spatial averaging method, and thereby suppress the covariance. This spatial averaging method is a method of obtaining a covariance matrix of the received signals, extracting a plurality of submatrices from the covariance matrix, averaging the submatrices to calculate a covariance submatrix, and calculating a weight coefficient from the covariance submatrix.

Moreover, in order to adaptively change the weight coefficient (weight vector), a degree of freedom is required to a certain extent. For example, when the matrix is of a 1 by 1 size during the calculation of the weight vector, the adaptability of changing the weight from the matrix information cannot be ensured. Accordingly, a certain number of latter input signals 023 is required in order to ensure the degree of freedom.

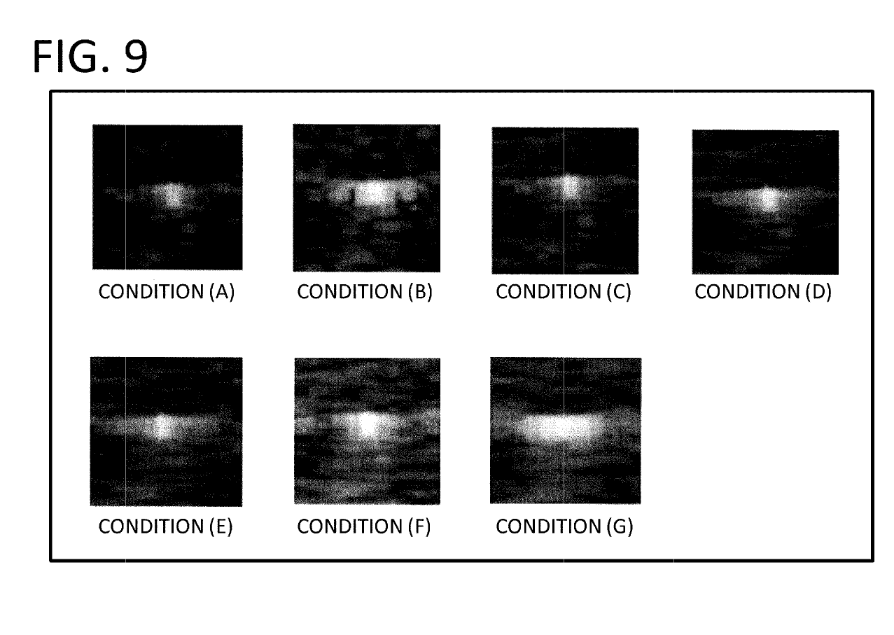

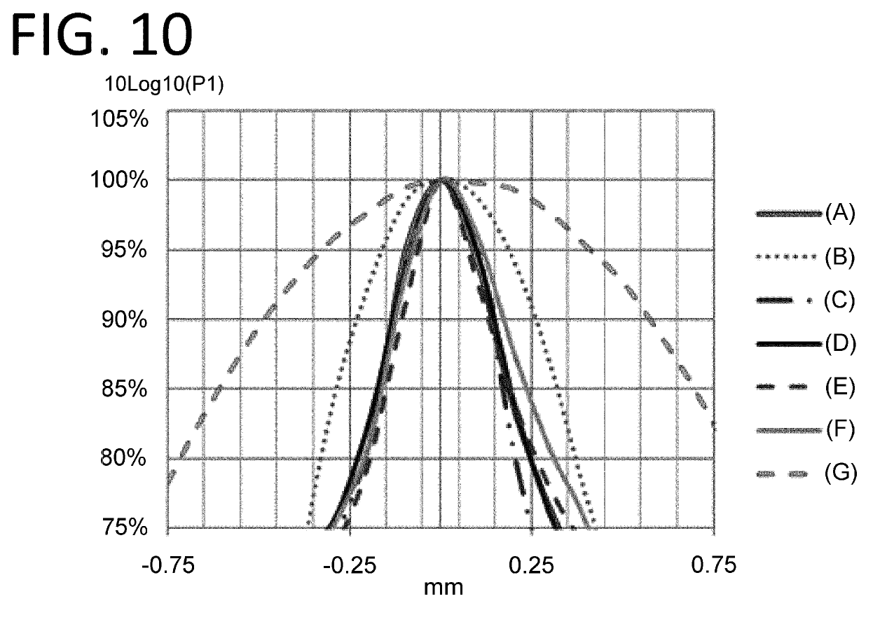

With respect to this point, the degree of freedom and adaptability are explained based on simulation results. FIG. 9 is a reconstruction of an image of a wire phantom (diameter of 0.1 mm) by using the delay-and-sum processing in the former stage and using the CAPON method in the latter stage. As the reconstruction method, a plurality of data among the received data of 32 elements were grouped, delay-and-sum processing was performed to the signals in that group to prepare latter input signals, and the latter input signals were processed with the CAPON method. FIG. 10 displays the cross section beam profile of the wire.

The number of latter input signals 023 is obtained by dividing 32, which is the number of received signals, by the number of elements of the received data to be subject to the delay-and-sum processing in the former stage, and the spatial averaging method performs averaging so as to achieve a number of rows which is half the number of the latter input signals 023. In other words, when the number of the former delay-and-sum is 4, the number of latter input signals 023 will be 8, and a matrix of 4 by 4 will be processed by the spatial averaging method. Nevertheless, with respect to the number of times spatial averaging is performed, the results will not change significantly even when the number of times that averaging is to be performed is changed.

Conditions (A) to (G) shown in FIG. 9 and FIG. 10 are now explained.

With condition (A), the CAPON method was performed using all 32 signals.

With condition (B), the delay-and-sum processing was performed using all 32 signals.

With condition (C), the delay-and-sum was performed using former 2 elements, and the CAPON method was performed using latter 16 inputs.

With condition (D), the delay-and-sum was performed using former 4 elements, and the CAPON method was performed using latter 8 inputs.

With condition (E), the delay-and-sum was performed using former 8 elements, and the CAPON method was performed using latter 4 inputs.

With condition (F), the delay-and-sum was performed using former 10 elements, and the CAPON method was performed using latter 3 inputs.

With condition (G), the delay-and-sum was performed using former 16 elements, and the CAPON method was performed using latter 2 inputs.

Foremost, with condition (A), the beam width became narrower than condition (B), and this shows the basic effect of the CAPON method. Moreover, from condition (C) to condition (F), it was confirmed that the same level of azimuth resolution as condition (A) was obtained.

Nevertheless, with condition (G), the beam width spreads drastically. This is because the number of latter input signals 023 was limited to 2, and, since a matrix of 1 by 1 was processed via spatial averaging, the degree of freedom during the calculation of the weight coefficient was reduced excessively. Based on these simulation results, it can be acknowledged that a sufficient azimuth resolution can be obtained if the matrix is at least 2 by 2 during the calculation of the weight coefficient. In other words, with the CAPON method in ultrasound waves that require spatial averaging, the effect of the adaptive signal processing cannot be obtained unless the number of latter input signals 023 is made to be 3 or more.

As a result of obtaining the value of the inverse matrix Rxx⁻¹ or the like by using the foregoing rule after performing the spatial averaging, the intensity of the output power Pout is ultimately calculated. The result of processing this power value in a time series becomes the image scanning line data in the intended signal direction at the arbitrary position. As described above, by performing the foregoing series of processing by moving the transmitting/receiving apertures on the array, two-dimensional ultrasound image data is output. Note that, although the linear scanning processing was explained above, the present invention can also be applied to convex scanning, sector scanning and radial scanning in addition to linear scanning by homologizing the direction of the intended signals described above. Moreover, in addition to 1D and 1.5D probes, this method is also effective for 2D array probes.

### Embodiment 1

Embodiments of the present invention are now explained in light of the above.

Embodiment 1 explains an ultrasound imaging apparatus that uses the delay-and-sum processing in the former stage and uses the adaptive signal processing (CAPON method) in the latter stage.

### (Configuration of Ultrasound Device)

FIG. 1 is a schematic diagram of the system of the ultrasound device of this embodiment. The ultrasound device comprises a transmitting circuit processing system 001, a receiving circuit processing system 002, a system controller 003, an ultrasound probe 004, an adaptive signal processor 006, an image processor 007, and an image display unit 008. The ultrasound probe 004 comprises a plurality of conversion elements (transducers) 005.

Moreover, FIG. 5 shows the details of the receiving circuit processing system 002 and the adaptive signal processor 006. The receiving circuit processing system 002 is configured from a delay unit 009 and a plurality of sum processors 011. The adaptive signal processor 006 is configured from a Hilbert transformer 012, a covariance matrix calculator 013, a spatial smoothing calculator 014, and an electric power calculator 015. The Hilbert transformer corresponds to a complex signal converter.

### (Operation of Ultrasound Device)

When the position (focus position) to which the ultrasound waves are to be transmitted is set, the setup information thereof is sent from the system controller 003 to the transmitting circuit processing system 001 illustrated in FIG. 1. Based on the foregoing information, the transmitting circuit processing system 001 decides the elements to transmit the ultrasound waves and their respective delay times. Subsequently, the transmitting circuit processing system 001 sends electrical signals for driving the corresponding conversion elements 005 in the ultrasound probe 004. These electrical signals are converted into displacement signals by the conversion element 005 and then propagated as ultrasound waves toward the object.

The ultrasound waves that were transmitted and propagated as described above are reflected or scattered by the object and once again return to the conversion elements 005 as ultrasound echoes. As a result of the ultrasound echoes being converted into electrical signals (received signals) in the 32 conversion elements 005 forming the receiving apertures among the above, the biological information of the object can be acquired as 32 electrical signals. These received signals are sent to the receiving circuit processing system 002. The receiving circuit processing system 002 determines the delay time of the received signals based on the depth information of the received data, and performs delay processing on the respective received signals. This delay processing is performed in the delay unit 009 in the receiving circuit processing system 002 illustrated in FIG. 5, and processing is performed so as to match the phases of the signals arising from the ultrasound echoes from the object that were received by the respective conversion elements 005. Note that the same processor may be diverted to the respective delay units for transmissions and receptions. The respective signals that were subject to the delay processing are combined for every 4 channels, which is a number that is set in advance, and sent to the respective signal adders 011. The signals of the 4 channels with matched phases are added in the signal adder 011, and the latter input signals 023 combined into 8 channels are sent to the adaptive signal processor 006.

In the adaptive signal processor 006, foremost, the Hilbert transformer 012 transforms the respective latter input signals 023 of the 8 channels into complex signals, whereby an 8-channel complex vector is created. In the covariance matrix calculator 013, a complex covariance matrix of a 4 by 4 size is calculated. Subsequently, in the spatial averaging unit 014, the complex covariance matrix is averaged into a covariance submatrix of a 4 by 4 size. In the electric power calculator 015, the weight vector is adaptively calculated from the matrix obtained by the spatial averaging unit and the weight and the direction of the designated intended signals, and that weight vector is used to generate an output power Pout. Note that, in the delay unit 009, if the amount of delay is caused to coincide in all 32 channels and not for every 4 channels, there is no need to seta vector for designating the direction of the intended signals. Nevertheless, if the phases of the respective latter input signals 023 of the 8 channels that were subject to delay-and-sum have not been matched, then it is necessary to calculate the intensity of the output power Pout by using a constrained vector which designates the direction of the respective intended signals.

The output power Pout is sent to the image processor 007, and subject to processing such as LOG compression so as to create image scanning line data. As a result of performing the foregoing series of processing for each image scanning line, a two-dimensional ultrasound image is created. The ultrasound image created by the image processor 007 is sent to the image display unit 008 such as an LCD, and the image is thereby visibly displayed. Here, the image display unit 008 is not limited to an LCD, and other image display units such as a CRT, a PDP, an FED, or an OLED may also be used.

The main signal flow is as described above.

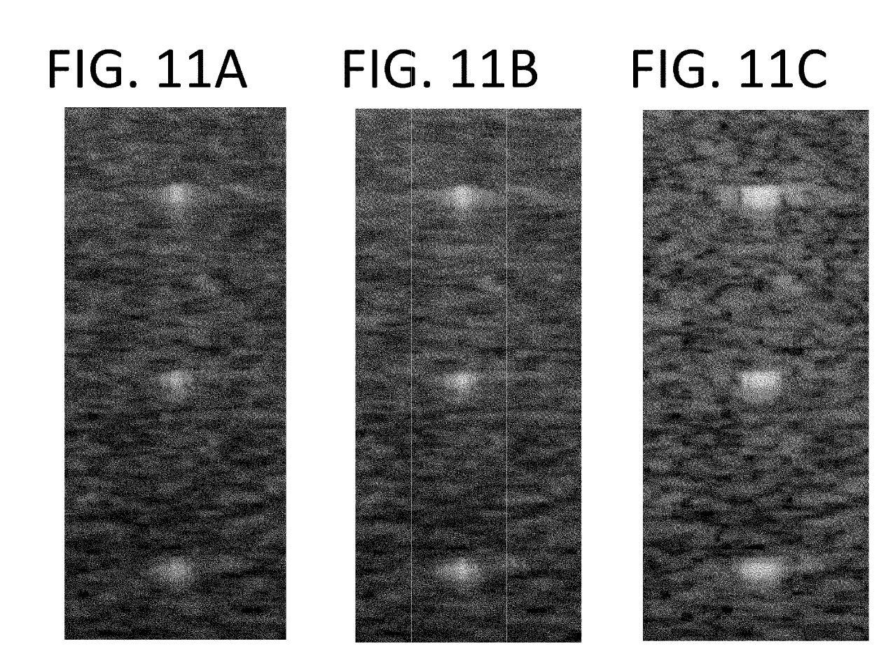

The results of reconstructing a wire (diameter of 0.1 mm) image in a phantom for ultrasound waves based on the foregoing system are shown in FIG. 11B. By way of comparison, the image in which 32 signals were reconstructed with the CAPON method is shown in FIG. 11A, and the image in which 32 signals were reconstructed with the delay-and-sum processing is shown in FIG. 11C.

The beam width in the orientation direction at roughly −1.94 dB of the TOP value of these wire images was 0.5 mm in FIG. 11A, 0.5 mm in FIG. 11B, and 0.75 mm in FIG. 11C. Consequently, it was possible to confirm that the image quality yielded a favorable azimuth resolution in comparison to the case of only performing the delay-and-sum processing, and that an image quality that is in no way inferior to the case of only performing the adaptive signal processing could be realized. Moreover, it was possible to reduce the processing volume of this embodiment to roughly 1/64 in comparison to the case shown in FIG. 11A.

Note that, in this embodiment, the processing was performed with 32 as the number of input signals, 4 as the number of times the former delay-and-sum processing is performed, 8 as the number of latter input signals, and 4 by 4 as the vector size after the spatial averaging, but other values may be used instead of the foregoing values.

### Embodiment 2

### Configuration of Ultrasound Device

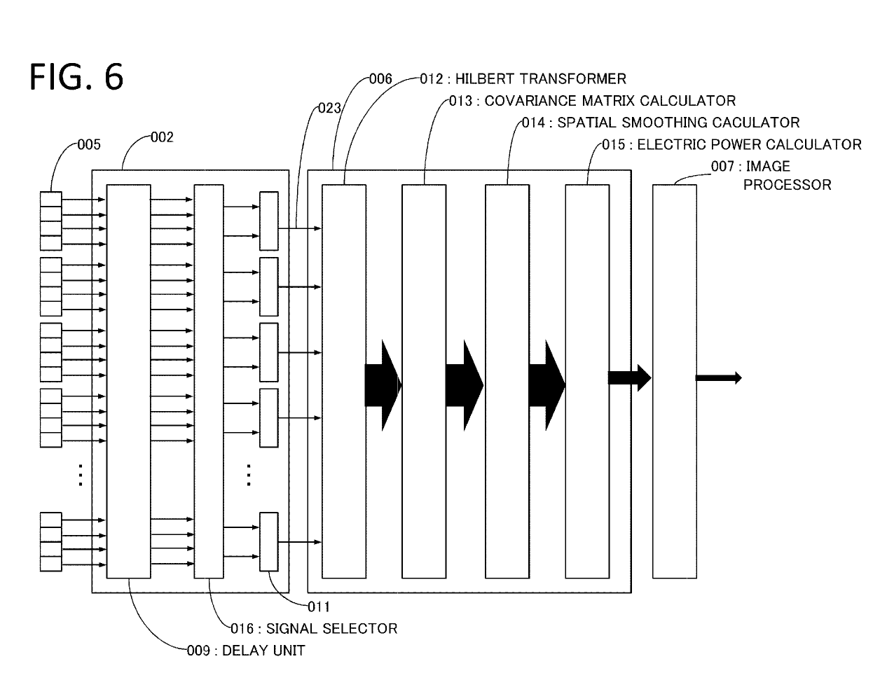

Embodiment 2 of the present invention is now explained. In this embodiment, the system of FIG. 1 explained in Embodiment 1 may be used, but different processing is performed in the receiving circuit processing system 002. FIG. 6 shows the configuration of this embodiment, and a signal selector 016 is provided between the delay unit 009 and the sum processor 011 illustrated in FIG. 5 of Embodiment 1.

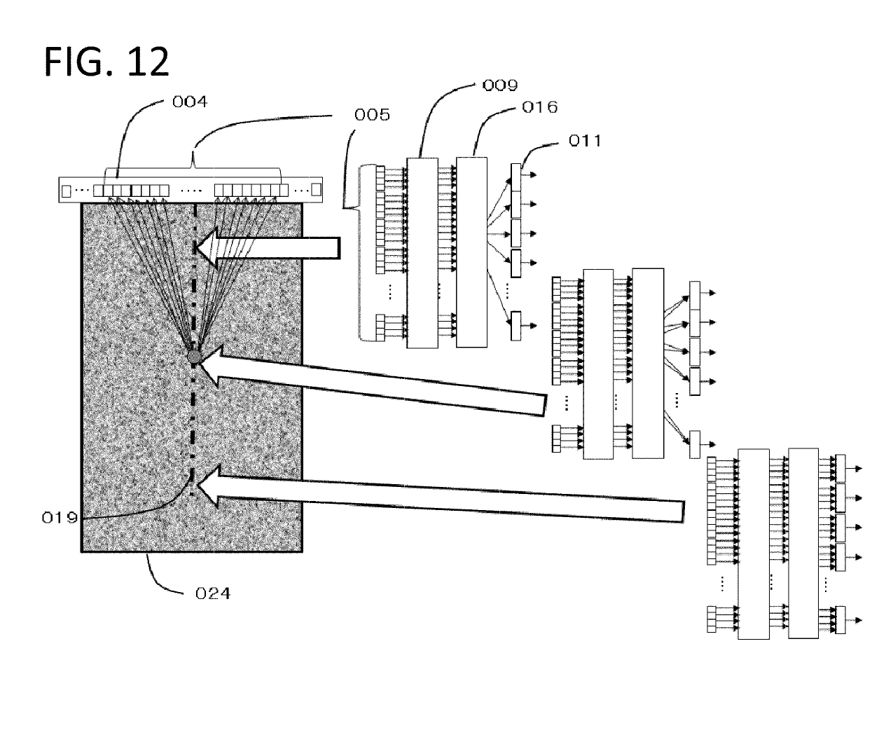

Moreover, FIG. 12 is a diagram for explaining this embodiment in correspondence with aperture control, and shows the relationship of the target depth on the image scanning line 019 in the object 024 and the operation of the signal selector 016.

### (Operation of Ultrasound Device)

In the image reconstruction of ultrasound waves, when the depth to be measured is small; that is, when calculating the intensity of the output power of a position in which the distance from the conversion elements is relatively close, aperture control is sometimes performed to suppress a side lobe. Aperture control is the processing of adjusting the number of the plurality of transmitting conversion elements 005 and receiving conversion elements 005 configuring the transmitting/receiving apertures of the ultrasound wave in correspondence with the measured depth, and the number of transmitting/receiving elements is decreased as the measured depth becomes smaller. The decrease of the number of elements is not achieved by generally thinning out the conversion elements, and is achieved by narrowing the range of the conversion elements. Note that, since the performance of aperture control is irrelevant with the transmitted signals of the present invention, only the subject matter concerning the received signals is explained.

The received signals that were converted into electrical signals by the conversion elements 005 illustrated in FIG. 6 are sent to the delay unit 009 of the receiving circuit processing system 002, and delay processing is performed based on the signals from the system controller 003. The respective signals that were subject to delay processing are sent to the signal selector 016. The signal selector 016 allocates all received signals to the signal adder 011 divided into the latter 8 blocks based on the selected signals sent from the system controller 003. Moreover, in this processing, it is also possible to refrain from allocating the signals from the arbitrary conversion element 005 anywhere. In this embodiment, the number of conversion elements 005 used for the synthesis processing is reduced as 32, 24, 16, 8 as the object depth becomes smaller, and evenly sorted to the 8 signal adders 011. The latter input signals 023 of 8 channels are created in the signal adder 011, and, by subsequently performing the same processing as Embodiment 1, image is reconstructed. Note that the same processor may be diverted to the respective delay units for transmissions and receptions.

As a result of performing the foregoing processing, as shown in FIG. 12, the number of conversion elements 005 to be referred to in the synthesis processing can be adjusted in correspondence with the target depth on the image scanning line 019, whereby aperture control is enabled. With respect to the depth where aperture control is to be performed or the number of conversion elements according to the depth, a predetermined number of conversion elements corresponding to the range of a predetermined depth may be set forth in advance according to the characteristics of the probe or the conversion elements, the accuracy of image reconstruction, and the type of object. Otherwise, the number of conversion elements can be adjusted by experimentally performing measurement and image reconstruction. The range of the predetermined depth and the number of conversion elements corresponding thereto may be stored in a memory or the like and referred to during image reconstruction.

As a result of introducing the foregoing method, it was possible to reduce the processing volume to roughly 1/64 in comparison to the case of processing all signals based on the CAPON method. Moreover, the image quality yielded a favorable azimuth resolution in comparison to the case of only performing the delay-and-sum processing, an image quality that is in no way inferior to the case of only performing the CAPON method was realized, and the operation of aperture control was also realized.

Note that, as the number of input signals, the variation pattern of the number of signals to be sorted to the signal adders 011 in correspondence with the measured depth, and 8 as the number of signals to be combined, values other than those described above may be used.

### Embodiment 3

### Configuration of Ultrasound Device

Embodiment 3 of the present invention is now explained. This embodiment relates to an ultrasound device which absorbs the difference in the pin arrangement or number of element channels of the ultrasound probe based on probe information.

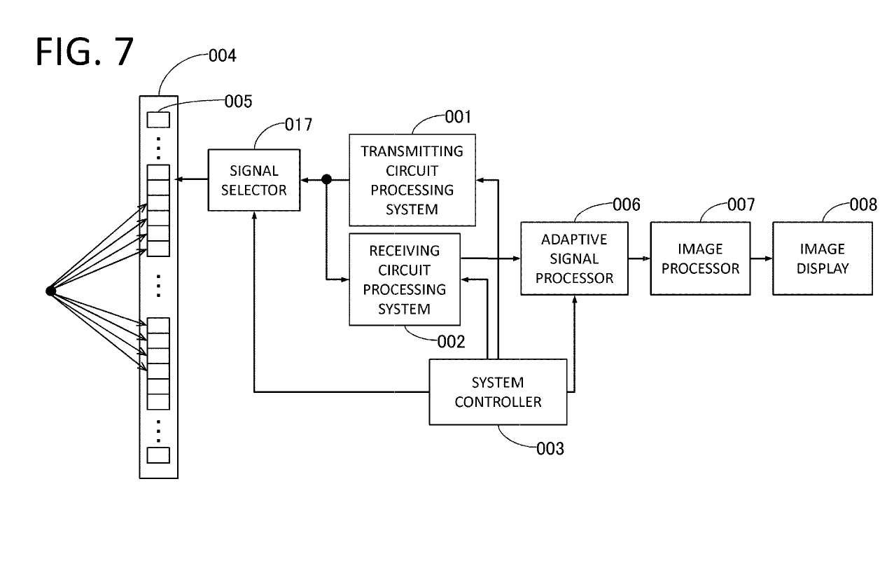

FIG. 7 shows the device configuration of this embodiment, and a signal selector 017 is provided between the transmitting circuit processing system 001/receiving circuit processing system 002 and the ultrasound probe 004 in the system shown in FIG. 1 of Embodiment 1. Note that the details of the receiving circuit processing system 002 and the adaptive signal processor 006 in this embodiment are the same as Embodiment 1 and are as shown in FIG. 5.

### (Operation of Ultrasound Device)

When the focus position or the information of the ultrasound probe 004 being used is set, the setup information thereof is sent from the system controller 003 to the transmitting circuit processing system 001. The transmitting circuit processing system 001 determines the type of transmitting elements and the respective delays times to be sent to those elements based on the foregoing information, and sends, to the signal selector 017, the electric signals for driving the corresponding conversion elements 005 in the ultrasound probe 004. The signal selector 017 sends the electrical signals to each of the corresponding conversion elements 005 based on the information of the ultrasound probe 004 or the depth information of the ultrasound signals sent from the system controller 001. In the conversion element 005, the sent electrical signals are converted into displacement signals, and propagated to the object as ultrasound waves.

Subsequently, the ultrasound echoes reflected off the object are received by the respective conversion elements 005, and acquired as electrical signals. The received signals are once again sent to the signal selector 017. The signal selector 017 chooses the received signals based on the information of the ultrasound probe 004 or the depth information sent from the system controller 001, and sends them to the delay unit 009 (FIG. 5) in the receiving circuit processing system 002. The delay unit performs delay processing only to the sent signals. The respective signals subject to delay processing are sent to a pre-set sum processor 011 (FIG. 5). Note that the same processor may be diverted to the respective delay units for transmissions and receptions. As a result of subsequently performing the same processing as Embodiment 1, the image is reconstructed.

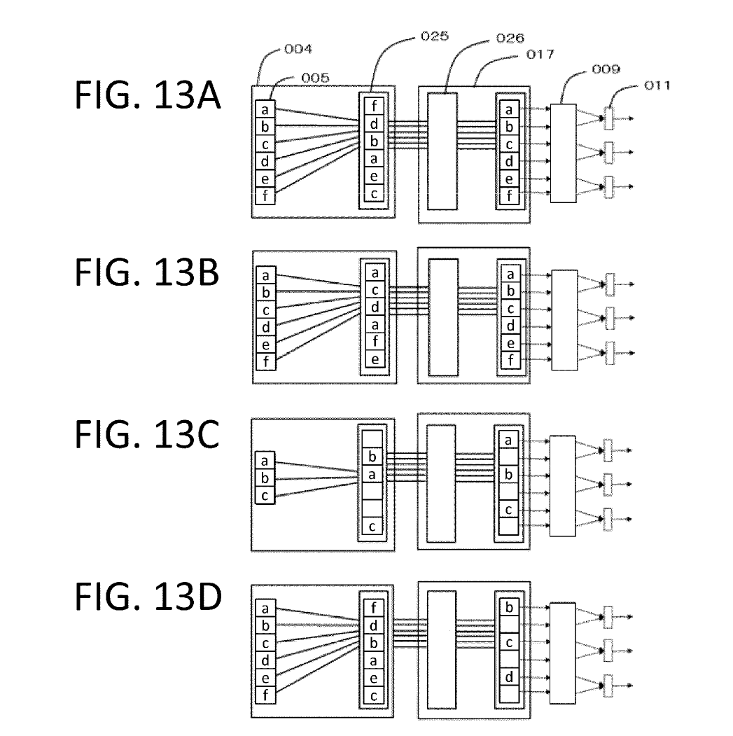

The function of the signal selector 017 is now explained with reference to FIG. 13.

FIG. 13 shows the ultrasound probe 004, the conversion element 005, the signal selector 017, and the receiving circuit processing system 002 of FIG. 7. Generally speaking, the ultrasound probe 004 is connected with the ultrasound imaging apparatus body via the probe connector 025, and the arrangement of the pins of the probe connector corresponding to the respective conversion elements 005 will differ depending on type of probe. The signal selector 017 comprises a selector switch 026 for sorting the signals.

FIG. 13A and FIG. 13B are used to explain the operations of respectively using the ultrasound probes 004 in which the number of conversion elements 005 is the same but the pin arrangement of the probe connector 025 is different. When the foregoing two types of ultrasound probes 004 are used in a state with no selector switch 026, the respective conversion elements 005 and the input/output channels of the corresponding delay units will not match. As a result of acquiring probe information from the system controller 003 in advance, the selector switch rearranges the pin arrangement so as to attain a match. Consequently, the two types of ultrasound probes can perform the same processing.

Specifically, two linear probes of 128 channels having different pin arrangements of the connector were prepared for confirmation. Since the band of the respective probes and the sensitivity of the respective elements are different, it is not possible to obtain an image that is completely the same, but it was confirmed that a high resolution image using the CAPON method in the same system can be reconstructed.

Subsequently, FIGS. 13A and 13C are used to explain the operations of using the ultrasound probes 004 having a different number of conversion elements 005 and a different pin arrangement of the probe connector 025. As a result of acquiring probe information from the system controller 003 in advance, the selector switch rearranges the pin arrangement to a setting corresponding to the latter synthesis processing. Even in cases where the number of signals for synthesizing an image is different, it is possible to evenly sort the signals to the sum processors 011, and output an image.

Specifically, linear probes of 256 channels and 128 channels were prepared for confirmation. Since the number of elements forming the transmitting/receiving apertures, bands, and sensitivity of the respective elements in the respective probes are different, it is not possible to obtain an image that is completely the same, but it was confirmed that a high resolution image using the CAPON method in the same system can be reconstructed.

Finally, FIGS. 13A and 13D are used to explain the operation corresponding to aperture control. FIG. 13A corresponds to processing of a deep part of the object and FIG. 13D corresponds to processing of a shallow part of the object. As a result of acquiring probe information from the system controller 003 in advance, the selector switch rearranges the pin arrangement to a setting corresponding to the latter synthesis processing. The number of latter input signals 023 of the probes become equal, and FIG. 13D can maximize the effects of the adaptive signal processing, and perform the same processing as Embodiment 2.

As a result of performing this embodiment, an image can be reconstructed with the same processing system even in cases of using ultrasound probes having a different number of channels and a different pin arrangement, and it is possible to deal with situations of changing the number of elements in aperture control or the like.

Specifically, as the measured depth became smaller in the linear probe of 128 channels, the number of transmitted signals was changed as 32, 24, 16, 8 based on signal switching. Consequently, it was possible to realize a high resolution image using the CAPON method in the same system, and also realize the operation of aperture control.

### Embodiment 4

### Configuration of Ultrasound Device

Embodiment 4 of the present invention is now explained. This embodiment relates to a photoacoustic imaging apparatus which receives photoacoustic signals (photoacoustic waves), and performs image reconstruction based on adaptive signal processing.

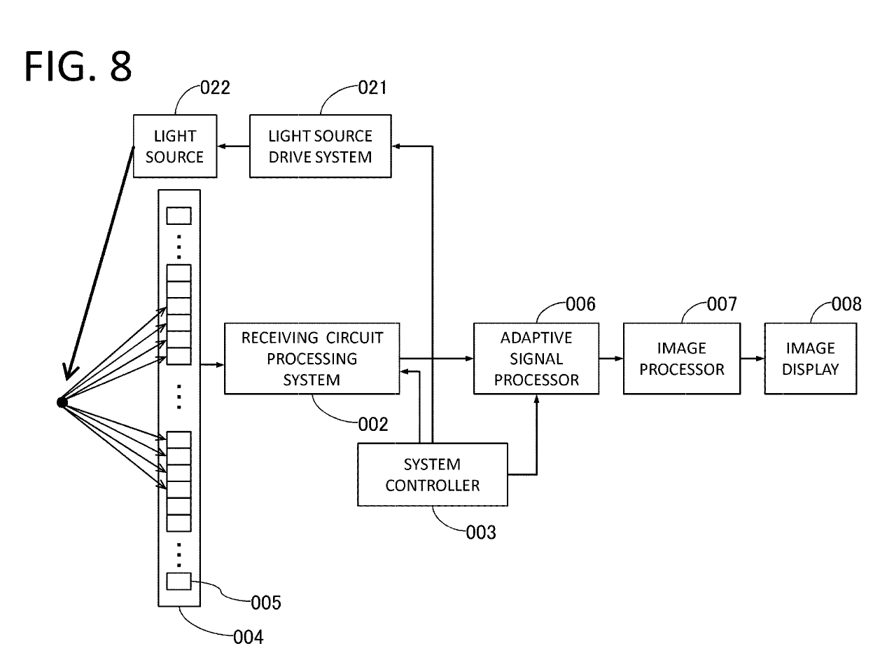

FIG. 8 shows the device configuration of this embodiment, and a light source drive system 021 and a light source 022 are provided in substitute for the transmitting circuit processing system 001 of the system shown in FIG. 1 of Embodiment 1.

### (Operation of Ultrasound Device)

When the target position in the object is set, the setup information thereof is sent from the system controller 003 to the light source drive system 021 illustrated in FIG. 8. The light source drive system 021 drives the light source 022 based on the foregoing setup information, and irradiates the object with pulsed electromagnetic waves such as pulsed laser. The acoustic waves emitted from within the object based on the foregoing irradiation are received by the conversion element 005. As a result of subsequently performing the same processing as Embodiment 1, the image is reconstructed.

As a result of introducing the foregoing method, the input signals are combined every 4 signals, and it was possible to reduce the processing volume to roughly 1/64 in comparison to the case of processing all signals based on the CAPON method.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-105317, filed on May 10, 2011, which is hereby incorporated by reference herein in its entirety.

1. An object information acquiring apparatus, comprising:
a probe including a plurality of conversion elements which receive acoustic waves emitted from an object and convert the acoustic waves into received signals;
a delay unit which matches phases of the plurality of received signals output from said plurality of conversion elements;
a signal adder which groups the plurality of received signals output from said delay unit, and adds the received signals for each of the groups to obtain latter input signals; and
an adaptive signal processor which generates internal image data of the object by performing adaptive signal processing on the plurality of latter input signals output from said signal adder.

2. The object information acquiring apparatus according to claim 1, wherein said adaptive signal processor includes:
a complex signal converter which converts the plurality of latter input signals into complex signals;
a covariance matrix calculator which calculates a covariance matrix from the plurality of complex signals output from said complex signal converter;
a spatial smoothing calculator which extracts a plurality of submatrices from the covariance matrix and averages the submatrices to calculate a covariance submatrix, and obtains a weight coefficient from the covariance submatrix; and
an electric power calculator which obtains an output power of the received signal by using the weight coefficient.

3. The object information acquiring apparatus according to claim 2, wherein said adaptive signal processor performs the adaptive signal processing by using a CAPON method.

4. The object information acquiring apparatus according to claim 1, wherein said signal adder groups the plurality of received signals into at least three groups and then adds the received signals, respectively, to obtain the latter input signals.

5. The object information acquiring apparatus according to claim 2, wherein said signal adder narrows the range of the conversion elements used for calculating the output power as the depth from the probe at the position inside the object where the output power is obtained becomes smaller.

6. The object information acquiring apparatus according to claim 1, wherein said probe includes a connector for connecting said plurality of conversion elements and said delay unit, and
wherein said object information acquiring apparatus further comprises a signal selector which controls said connector to match channels of said delay unit and said conversion elements.

7. The object information acquiring apparatus according to claim 1, wherein the acoustic waves emitted from the object result from the acoustic waves output from said conversion elements being reflected inside the object.

8. The object information acquiring apparatus according to claim 1, further comprising:
a light source which irradiates the object with electromagnetic waves,
wherein the acoustic waves emitted from the object are photoacoustic waves emitted from the object irradiated with the electromagnetic waves.
